(19) United States
(12) Patent Application Publication
(10) Pub. No.: US 2025/0010000 A1
(43) Pub. Date: Jan. 9, 2025

(54) SYSTEM FOR SUPPORTING THE PULMONARY GAS EXCHANGE IN PATIENTS

(71) Applicant: GRÜNDLER GMBH, Freudenstadt (DE)

(72) Inventors: Markus GRÜNDLER, Freudenstadt (DE); Christoph GRÜNDLER, Freudenstadt (DE); Bernd RESLO, Landsberg am Lech (DE); Thilo JOOST, Friedberg (Hessen) (DE); Rainer KÖBRICH, Neulußheim (DE)

(73) Assignee: GRÜNDLER GMBH, Freudenstadt (DE)

(21) Appl. No.: 18/708,826

(22) PCT Filed: Nov. 10, 2022
(86) PCT No.: PCT/DE2022/000111

§ 371 Date: May 9, 2024

(30) Foreign Application Priority Data
Nov. 14, 2021 (DE) 10 2021 005 641.8

(51) Int. Cl. A61M 16/00; A61M 16/04; A61M 16/06

(52) U.S. Cl. CPC A61M 16/0057; A61M 16/022; A61M 16/04; A61M 16/0683; A61M 2016/0027

(57) ABSTRACT

A system for supporting pulmonary gas exchange in patients, including a ventilating system that has a filter in a line system of the ventilating system and is coupled to a dead space minimization system. To be able to use such a system, in particular also when the patients are children, the second of two pump units is fluidically connected in a region between the filter and a patient interface.

The invention relates to a system for supporting pulmonary gas exchange in patients having the features of the preamble of claim 1.

A system for supporting pulmonary gas exchange in ventilated patients is known from the publication DE 10 2017 006 655 A1. The publication describes complementation of a ventilating system, as it has been in use for many years, with a dead space minimization system. This dead space minimization system comprises two pump units, wherein one of the two pump units comprises a flexible hose that is inserted via an interface into the tracheal cannula or the endotracheal tube of the ventilating system, deep into the trachea of the patient. Breathing gas is aspirated end-expiratorily by means of the second pump unit and is then recirculated again at the beginning of expiration. A first of the two pump units of the dead space minimization system is connected, on the patient side, upstream from the Y-piece of the ventilating system via a further flexible hose and carries out the aspiration or pumping slightly staggered in time, but in any case in a manner opposite the second pump unit. The dead space minimization system has the effect that CO2-rich spent breathing gas flows better from the patient to the ventilating device, and oxygen-rich or low-CO2 fresh air flows better from the ventilating device to the patient.

Such a system, however, can only be used with limitations in smaller patients, for example children, since the ventilation hose of the ventilating system extending into the trachea of the patient accommodates the flexible hose of the second pump unit. Even though it is possible to design smaller hoses for children, in order to conduct sufficient volume flows, it is still necessary to have minimum cross-sections for the flexible hose and of the hose of the ventilating system, which may still be too large for smaller children. In particular due to the mere reduction of the cross-section, but also due to the shape and undefined position, the flexible hose creates considerable flow resistance in the ventilation hose, so that there may be instances where a dead space minimization would be desirable, but additional resistance in the ventilation hose is not acceptable from a medical perspective. These are set-ups, for example, in which the dead space portion of the ventilating system located outside the patient accounts for a relevant portion, for example when using so-called heat and moisture exchanger filters (HME filters, HMEFs), and such a filter is necessary, for example, to protect the treating staff against infection.

It is therefore the object of the invention to create a system for supporting pulmonary gas exchange that is, in particular, better suited for children and that allows the use of a filter, without the drawback of thereby increasing the dead space.

This object is achieved according to the invention by the system having the features of claim 1. The system according to the invention is used to support the pulmonary gas exchange in patients, wherein the patients can, in particular, be children, but can also be adults. It is also possible to treat animals.

The system comprises a ventilating system, which includes a ventilating device, a Y-piece, a filter, and a patient interface. In addition to a customary Y-shaped junction, “Y-piece” also refers to other elements having the same function, such as, for example, a coaxial hose having an integrated Y-connection. The ventilating system can moreover comprise further components, such as fluidic connecting elements. The filter is, in particular, a filter having an HME function. The patient interface is the interface between the ventilating system and the patient, which is to say, in the case of invasive ventilation, in particular a tracheal cannula or an endotracheal tube or, in the case of non-invasive ventilation, in particular a face mask or a ventilation helmet.

The system furthermore comprises a dead space minimization system that is fluidically coupled to the ventilating system. In particular, this is a dead space minimization system as is comprehensively described for ventilated patients in the publication DE 10 2017 006 655 A1. If any deviations exist, this will be pointed out hereafter.

The dead space minimization system comprises two separately controllable pump units, each including a reservoir unit. The pump units and reservoir units can, in particular, be jointly formed by a piston pump. The fluidic connection to the ventilating system is, in particular, established via flexible hoses.

The dead space minimization system also comprises a control unit, so that expiratory, in particular end-expiratory, aspiration of breathing gas and inspiratory, in particular end-inspiratory, or beginning-expiratory recirculation of the aspirated breathing gas by a second of the two pump units can be set, and the first of the two pump units essentially operates in a manner opposite the second pump unit. Since both pump units are fluidically coupled to the ventilating system, this has the advantage, as described in the publication DE 10 2017 006 655 A1, that no disruptive changes in the pressure and flow profiles through the system occur in the region of the ventilating system. The system according to the invention thus remains “invisible” to the ventilating system, and the ventilating system can be operated without adaptations. “Essentially in a manner opposite” encompasses that deviations from an exactly opposite operation occur. These are also desirable, at least in certain operating modes, which is only possible by separately controllable pump units.

The first pump unit is fluidically connected to the ventilating system in the region between the ventilating device and the filter, in particular between the Y-piece and the filter. “Fluidically connected” means that a fluidic flow, in particular a gas flow, is possible at this location from the ventilating system into the dead space minimization system, and vice versa.

The system according to the invention is characterized in that the second pump unit is fluidically connected in the region between the filter and the patient interface, and thus a hose of the dead space minimization system need not be guided through the trachea. As a result, no hose at all must be guided in the trachea in the case of a face or laryngeal mask, and a smaller hose cross-section can be selected with a tracheal cannula, an endotracheal tube or a laryngeal tube, as is the case in the system described in the publication DE 10 2017 006 655 A1. This, in particular, allows the system to be used in children. “In the region between” here also encompasses a connection directly to the filter or to the patient interface, in particular in the case of a face or laryngeal mask or a ventilation helmet. Surprisingly, it was found that, even though the fluidic connection between the filter and the patient interface decreases the effect of the dead space minimization system, the decrease, is small, since the dead space minimization due to the system according to the invention is not limited to the dead space between the two fluidic connecting points. It has therefore been shown that part of the aspiration from the connection close to the patient also concerns gas that is located on the patient side of the connection close to the patient. As a result, it is also possible to use the filter in accordance with the invention.

In the case of non-invasive ventilation, which preferably takes place by way of a face mask or a ventilation helmet serving as the patient interface, the second pump unit, as an alternative, can be fluidically connected through the patient interface to the nasal or pharyngeal space of the patient. “Through” here shall also be understood to mean a passage in the region in which the patient interface bears against the body of the patient. Compared to the above-described variant (connection between the filter and the patient interface), aspiration and recirculation are carried out even closer to the patient by way of the dead space minimization system, which enhances the elimination by the dead space minimization system. However, a hose in the trachea is not required. Additionally, another advantage arises. During non-invasive ventilation, leakage losses frequently occur in the region of the seal of the patient interface with respect to the patient. These are compensated for by inflowing ventilation gas so as to maintain the desired pressure. Due to resulting dilution effects, measurements of the CO2 concentration in the exhalation flow are usually not suitable for determining the true CO2 content of the gas exhaled by the patient with sufficient precision. Due to the connection of the second pump unit to the nasal or pharyngeal space of the patient, the CO2 content of the breathing gas pumped by the second pump unit can be measured, and more particularly essentially without distortion by leakage effects. In this way, a reliable value for determining the end-expiratory CO2 partial pressure can be ascertained by way of the dead space minimization system.

The two described variants (connection between the filter and patient interface, or connection to the nasal or pharyngeal space of the patient) share the basic idea that it may suffice for an effective elimination of the dead space during ventilation when the second pump unit is not connected to the patient-side end of this dead space, namely in the lung, but within the overall distance of the dead space.

Even if the ventilating device were to be temporarily shut off and/or decoupled, the described connection of the second pump unit in the region of the nasal/pharyngeal space can make a valuable contribution to reducing dead space effects.

In a preferred embodiment, the ventilating system comprises an aspiration system between the filter and the patient interface. The aspiration system, in particular, serves to guide an aspiration hose through the patient interface into the trachea so as to aspirate secretions. The invention preferably provides that the second pump unit is fluidically connected in the region between the aspiration system and the patient interface. The aspiration system, similarly to the filter, is thus located between the connections of the pump units and is compensated for as dead space by the dead space minimization system.

The second pump unit is preferably fluidically connected to the ventilating system so that a gas flow from the first pump unit joins the ventilating system at an angle of 0° to 70°, and preferably between 30° and 60°. This is, in particular, ensured by a connector that has the shape of a pipe section having an oblique branch. By obliquely merging the gas flows, a loss of pressure due to turbulences compared to merging at a right angle is decreased, and thus the effect of the dead space minimization system is improved. In particular, the gas flow from the first pump unit joins obliquely toward the patient interface, especially during expiratory aspiration and inspiratory recirculation, since then the respective directions of the gas flows harmonize with one another.

In the case of beginning-expiratory recirculation, the fluidic connection of the second pump unit is such that a gas flow from the second pump unit joins the ventilating system so that the gas flow from the second pump unit, when joining, has a directional component parallel to a gas flow of the ventilating system toward the ventilating device.

For expiratory aspiration, the fluidic connection of the second pump unit is preferably such that a gas flow leaves the ventilating system toward the second pump unit so that the gas flow toward the second pump unit, when leaving, has a directional component parallel to a gas flow of the ventilating system toward the ventilating device.

In the case of a rigid fluidic connection, the two aforementioned conditions contradict one another. The invention therefore provides that the connection is carried out via alternating mouths, which is to say, a first mouth through which a gas flow from the second pump unit joins the ventilating system with a directional component parallel to a gas flow of the ventilating system toward the ventilating device, and a second mouth through which a gas flow leaves the ventilating system toward the second pump unit such that the gas flow toward the second pump unit, when leaving, has a directional component parallel to a gas flow of the ventilating system toward the ventilating device. Such alternating mouths can, in particular, be implemented via a junction and check valves between the junction and the mouths.

So as to improve the parallelity of the gas flows, the invention provides that the second pump unit is fluidically connected to the ventilating system so that a mouth for the gas flow from and to the second pump unit is formed by an appendage protruding into the interior of a connector. The connector is, in particular, a second connector. The appendage can be designed so that the open end thereof points in the flow direction toward the patient or toward the ventilating device. In particular, the appendage is a nozzle-like, curved pipe. The system can comprise only one such appendage, which in particular points in the flow direction toward the patient, or, in the above-described system having alternating mouths, can comprise two appendages, the open ends of which point in opposite directions.

As mentioned above, if non-invasive ventilation is carried out, this preferably takes place by way of a face mask or a ventilation helmet serving as the patient interface. It is preferred that the patient interface has a sealing, in particular multiply closable, through-passage including fixation means for a fluid connection between the nasal or pharyngeal space of the patient and the second pump unit. For example, a hose that is connected to the second pump unit can extend through an elastically closing opening, serving as a through-passage in a face mask, to the nasal or pharyngeal space of the patient.

As an alternative or in addition, it may be provided that the face mask or the ventilation helmet, serving as the patient interface, comprises an interface connector for connecting a fluid connection to the second pump unit as well as a patient hose for the fluidic connection to the nasal or pharyngeal space of the patient. The patient hose can be directly integrally formed on the interface connector on the patient side of the patient interface or be connected via a further connector as a separate component. In the second case, the interface connector and the further connector can be designed as a double-sided connector, so that a hose can be connected both on the patient side and on the environment side (hereafter “interior” and “exterior”). The connection can be established in the known manner, for example, by way of plugging-in or clamping or by means of magnetic coupling. Before the fluid connection is connected to the second pump unit, the interface connector can be closed by a protective cap. The patient hose can be flexurally rigid, at least in part of the length thereof, in particular close to the patient, which is to say, in particular can be configured, by an additional element or an additional material component, so as to be bendable by hand, while maintaining the shape thereof under the weight thereof.

The system preferably comprises a nose prong and/or an atraumatic catheter for the fluidic connection to the nasal or pharyngeal space of the patient. A “nose prong” shall be understood to mean a nasal prong, such as is known, for example, from high flow nasal prong (HFNP) oxygen therapy. An “atraumatic catheter” shall be understood to mean a catheter that, due to the design thereof, decreases the risk of attaching to the mucous membrane by suction and/or blockage due to secretions. For example, the catheter, similarly to an aspiration catheter, has several openings and/or an annular bead around the catheter tip for this purpose.

The invention will be described hereafter based on two exemplary embodiments. In the drawings:

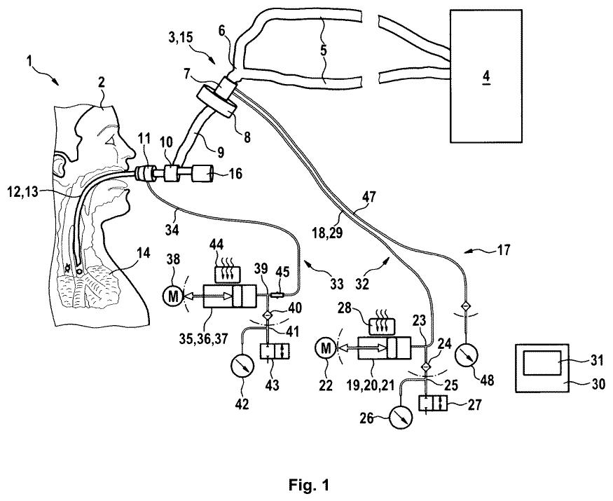

FIG. 1 shows a schematic illustration of the first exemplary embodiment of the system according to the invention while in use in a ventilated patient;

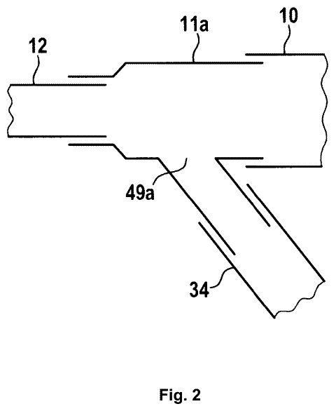

FIG. 2 shows a detail of this system in a schematic illustration in the region of a fluidic connection of the dead space minimization system to the ventilation system;

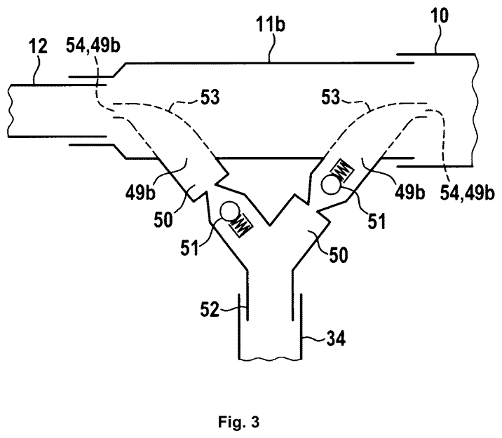

FIG. 3 shows a detail as in FIG. 2, however with an alternative fluidic connection and with a variant of the alternative connection;

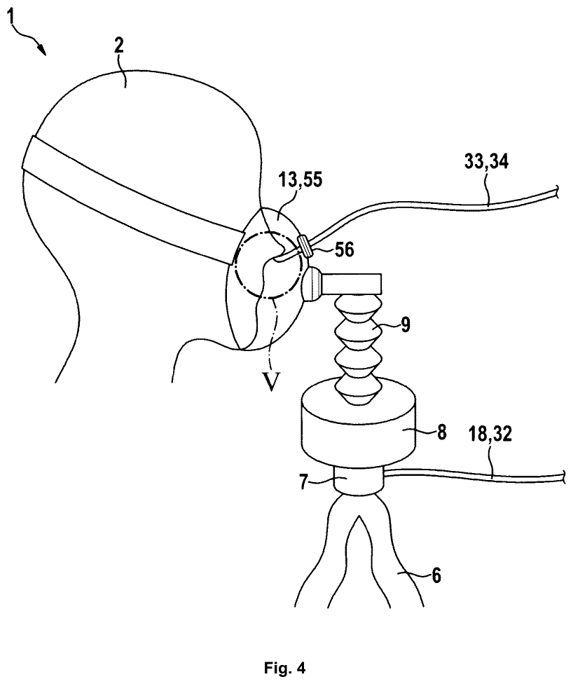

FIG. 4 shows a schematic illustration of the second exemplary embodiment;

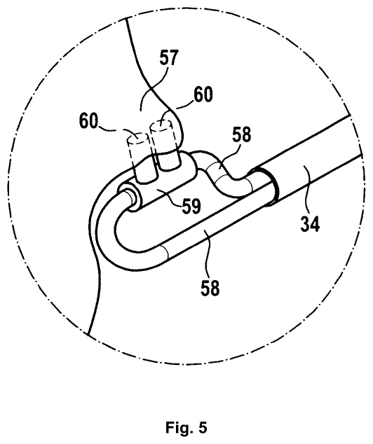

FIG. 5 shows a schematic detailed view of the detail from FIG. 4 denoted by V;

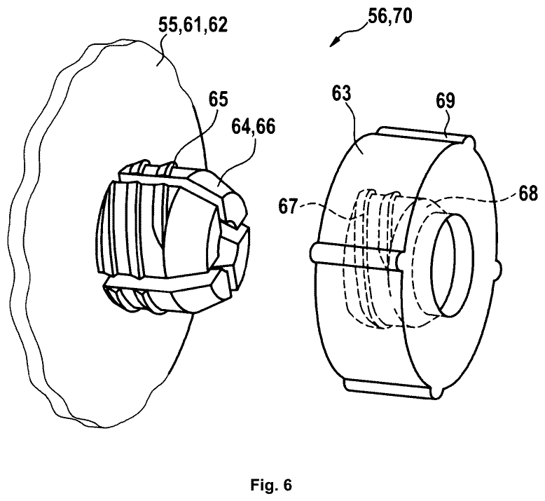

FIG. 6 shows an exemplary design of a through-passage for the second hose of the second exemplary embodiment; and

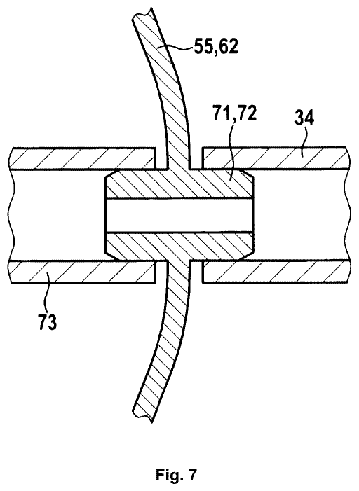

FIG. 7 shows an alternative for this through-passage.

FIG. 1 shows a schematic overview of a first exemplary embodiment of a system 1 according to the invention while in use in a ventilated patient 2.

The patient 2 is ventilated by way of a ventilating system 3. The ventilating system 3 comprises a ventilating device 4 including a pump, a control unit, a user interface, and the like, the details of which are not important here. Two ventilation hoses 5 extend from this ventilating device 4 to a Y-piece 6, which is followed in succession by a first connector 7, a filter 8 having HME function, a hose-like elongation 9, an aspiration connector 10, and finally a second connector 11. The extension 9 can also be interpreted as a tube extension. An endotracheal tube 12, serving as a patient interface 13, is attached to the second connector 11, which will be described in greater detail hereafter. The ventilating system 3 operates in the known manner, which is to say, air, oxygen and/or therapeutic gases are pumped into the lung 14 of the patient 2 via a ventilation hose 5, the endotracheal tube 12, and the interposed components and, in alternation therewith, the spent air is removed again in the reverse direction, but via the other ventilation hose 5. Together with the endotracheal tube 12 and the interposed components, the ventilation hoses 5 form a line system 15 of the ventilating system 3. An aspiration system 16, such as is known, for example, from the publication U.S. Pat. No. 7,779,842 B1, is connected via the aspiration connector 10 to the line system 15 of the ventilating system 3. The aspiration system 16 is only shown schematically in the figure since the details are not relevant.

In addition, a dead space minimization system 17 is connected to the ventilating system 3. The dead space minimization system 17 comprises a first hose 18, which is connected to the first connector 7. The first hose 18 is used for the fluidic connection to a first reservoir unit 19 of the dead space minimization system 17. The first reservoir unit 19 is designed as a first piston pump 20, and thus also forms a first pump unit 21. The first piston pump 20 is driven by a first linear motor 22, which is schematically illustrated in FIG. 1.

The first hose 18 is interrupted by a first branch 23. The first branch 23 leads via a first filter element 24 to a second branch 25, to the one outlet of which a first high pressure sensor 26 is connected, and to the other outlet of which a first aeration and bleed valve 27 having an outlet to the environment is connected. The piston pump 20 can be heated by a first thermoelectric heating element 28. Moreover, the first hose 18 is surrounded by a first thermal insulation 29.

The actuators, which is to say, the first linear motor 22, the first heating element 28, and the first aeration and bleed valve 27, are each connected to a control unit 30 via electrical lines, which are not shown for the sake of clarity. The control unit 30, in particular, comprises an input and output unit in the form of a touch-sensitive display 31, a so-called touch screen, to be operated by a user.

The first hose 18 including the first pump unit 21 and the components connected to the first branch 23 forms a first pathway 32 of the dead space minimization system 17.

A second pathway 33 of the dead space minimization system 17 is formed by a second hose 34, which is fluidically connected via the second connector 11 to the ventilating system 3, and also by a second reservoir unit 35, which is designed as a second piston pump 36 and thus forms a second pump unit 37, a second linear motor 38, a third branch 39, a second filter element 40, a fourth branch 41, a second high pressure sensor 42, a second aeration and bleed valve 43, as well as a second heating element 44. The design corresponds to that of the first pathway 32, however the second hose 34 is interrupted, on the patient side in relation to the third branch 39, by a measuring cuvette 45 for a gas analysis sensor. The actuators of the second pathway 33 as well as the sensor in the measuring cuvette 45 are also connected to the control unit 30.

The two pathways 32, 33 without the two linear motors 22, 38, the two high pressure sensors 26, 42, and the two aeration and bleed valves 27, 43 form a patient set 46 designed as a disposable product, which does not preclude the individual components from being able to be treated for repeat use, which is to say, in particular, cleaned and sterilized.

The dead space minimization system 17 additionally includes a third hose 47, which is connected to the first connector 7 and via which a ventilation pressure sensor 48 is connected to the ventilating system 3. The third hose 47 also forms part of the patient set 46.

The two pathways 32, 33 support the ventilation of the patient 2, which is essentially carried out by the ventilating system 3 by pumping off and recirculating, in alternation, by means of the two piston pumps 20, 36. Breathing gas spent at the end of the expiration from the second pathway 33 is suctioned out of a part of the ventilating system 3 close to the patient, while fresh gas is essentially recirculated from the first pathway 32 at the same time. The recirculating, in fact, already begins slightly earlier so that no pressure reduction occurs in the line system 15 of the ventilating system 3, which would suggest inhalation by the patient to the ventilating system 3. Due to aspiration, the spent CO2-rich gas is removed from the region of the ventilating system 3 close to the patient through the second pathway 33 and is replaced with fresh gas supplied via the first pathway 32.

The gas aspirated from the second pathway 33 is recirculated at the end of the subsequent inspiration, or at the beginning of the subsequent expiration, while fresh gas from the first pathway 32 is essentially pumped out of the ventilating system 3 at the same time. Reference is also made to the publication DE 10 2017 006 655 A1 with respect to the function of the dead space minimization system 17.

FIGS. 2 and 3 show the connection of the second hose 34 of the dead space minimization system 17 to the ventilating system 3 in two alternative configurations. While FIG. 1 shows the second connector 11 so that the gas flow of the dead space minimization system 17 perpendicularly joins the gas flow of the ventilating system 3, or leaves the same perpendicularly, the second connectors 11a, 11b in FIGS. 2 and 3 each provide for oblique joining or leaving. Generally speaking, the second connector 11, 11a, 11b can be interpreted as a pipe section having one or more branches for the second hose 34.

The second connector 11a in FIG. 2 has a mouth 49a extending obliquely toward the patient 2, namely in such a way that a gas flow from the second pump unit 37 joins the ventilating system 3 at an angle of approximately 50°. This can also be interpreted as an oblique branch. During expiratory aspiration, a gas flow leaves the ventilating system 3 toward the second pump unit 37 so that the gas flow toward the second pump unit 37, when leaving, has a directional component parallel to a gas flow of the ventilating system 3 toward the ventilating device 4. In other words, the gas flow is divided in a Y-shaped manner in this situation. This favors aspiration, since too little pressure loss occurs due to turbulences. However, the recirculation from the second pump 37 with a beginning-expiratory recirculation occurs obliquely against the gas flow of the ventilating system 3, which is not optimal. Since the aspiration assumes the more important role, this is still preferable over an oblique arrangement in the direction toward the ventilating device 4.

The second connector 11b in FIG. 3 solves this by having two mouths 49b, which are obliquely disposed opposite one another, which is to say, one obliquely toward the patient 2 and one obliquely toward the ventilating device 4. Connecting pipes 50 lead to the two mouths 49b, in each of which a check valve 51 is disposed, and which together join a connecting piece 52 in a Y-shaped manner. In other words, the connecting piece 52 has a junction in the two connecting pipes 50. The second hose 34 is connected to the connecting pieces 52. The check valves 51 are disposed opposite one another, and more particularly so that, during aspiration, a gas flow coming from the patient 2 is divided in a Y-shaped manner, and the gas flow to the second pump unit 37, when leaving the ventilating system 3, has a directional component parallel to a gas flow of the ventilating system 3 toward the ventilating device 4. During recirculation from the second pump unit 37, the check valves 51 cause the gas flow to merge with the gas flow of the ventilating system 3 via the other of the two mouths 49b. The fluidic connection of the dead space minimization system 17 is thus carried out via alternating mouths 49b. In the case of beginning-expiratory recirculation, this means that the gas flow coming from the second pump unit 37 has a directional component parallel to a gas flow of the ventilating system 3 toward the ventilating device 4. In this way, low-turbulence gas conduction is achieved both for aspiration and for recirculation.

Using dotted lines, FIG. 3 additionally shows a variant of the mouths 49b. Nozzle-like, curved appendages 53 extend from the connecting pipes 50 into the interior of the second connector 11b. Due to the curvature of the appendages 53, open ends 54 of the appendages 53 in each case point in opposite directions, namely in the direction of the endotracheal tube 12 on the one hand, and in the direction of the aspiration connector 10 on the other hand, thus forming the mouths 49b. In this way, the gas flows flowing to or from the second pump unit 37 not only have a parallel directional component with respect to the gas flow of the ventilating system 3, but are essentially entirely parallel thereto.

FIG. 4 shows a schematic sectional illustration of a second exemplary embodiment. Many elements are identical to those in the first exemplary embodiment, which is why, for example, the pump units are not illustrated, and the same reference numerals are used otherwise for identical or corresponding elements. A first pathway 32 of the dead space minimization system is connected to a first connector 7 of the ventilating system by way of a first hose 18, similarly to the first exemplary embodiment. In contrast to the first exemplary embodiment, however, a face mask 55 serves as the patient interface 13. As is customary with non-invasive ventilation, an extension 9 in the form of a flexible hose is connected to this face mask 55, which is followed by a filter 8, the first connector 7, and a Y-piece 6.

The face mask 55 has a through-passage 56 for a second hose 34 of a second pathway 33 of the dead space minimization system. The through-passage 56 will be addressed further hereafter. The second hose 34 extends through the through-passage 56 into the face mask 55, and from there onward to the nose 57 of the patient 2. This is shown in greater detail in FIG. 5. Within the face mask 55, the second hose 34 splits into two feed lines 58, which lead into a nasal prong 59 from two sides. This nasal prong has two outlets 60 extending into the nose 57, wherein air can flow past the outlets 60. The second pump unit is thus fluidically connected via the second hose 34, through the patient interface 13, to the nasal space of the patient 2 or is joined to the nasal space. The function corresponds to that of the first exemplary embodiment.

FIG. 6 shows an exemplary design of the through-passage 56. The through-passage 56 is made of two parts, this being a base body 61 that is integral with a mask base body 62 of the face mask 55 on the one hand, and a clamping collar 63 on the other hand. Both parts are substantially rotation-symmetrical. The base body 61 comprises a four way-slotted clamping piece 64, which has an external thread 65 and a conical end 66. The clamping collar 63 is sleeve-like and has an internal thread 67 that matches the external thread 65 and is followed by a conical diameter taper 68. The clamping collar 63 comprises four longitudinal beads 69, which are distributed over the outer circumference, for improved handling.

When the system 1 is attached to the patient 2, the second hose 34 is pushed through the base body 61 and the clamping collar 63 of the through-passage 56, from the interior of the face mask 55 to the outside. As soon as the nasal prong 59 and the face mask 55 have been fixed to the patient 2, the position of the second hose 34 can be fixed by means of the through-passage 56. For this purpose, the clamping collar 63 is screwed onto the clamping piece 64. The conical end 66, the diameter taper 68 and the slots of the clamping piece 64 together cause the clamping piece 64 to be radially compressed, whereby the second hose 34 is clamped. The slots are dimensioned such that sealing is achieved at the same time. The clamping piece 64 and the clamping collar 63 thus form a fixation means 70 for the second hose 34, and the through-passage 56 is sealed.

FIG. 7 shows an alternative to the through-passage 56. The face mask 55 here, in one piece, comprises a double connector 71 in the form of a pipe section, which extends through the mask base body 62 of the face mask 55 or extends therefrom to the inside and outside. The outer part of the double connector 71 forms an interface connector 72 onto which the second hose 34 is mounted. This could additionally be secured by way of a clamp, serving as a fixation means. On the inner side, a patient hose 73 is mounted onto the double connector 71. The patient hose 73, in turn, can be connected to a nasal prong or to an atraumatic catheter.

List of reference numerals

| | |
|---|---|
| 1 | system |
| 2 | patient |
| 3 | ventilating system |
| 4 | ventilating device |
| 5 | ventilation hose |
| 6 | Y-piece |
| 7 | first connector |
| 8 | filter |
| 9 | extension |
| 10 | aspiration connector |
| 11, 11a, 11b | second connector |
| 12 | endotracheal tube |
| 13 | patient interface |
| 14 | lung of the patient 2 |
| 15 | line system of the ventilating system 3 |
| 16 | aspiration system |
| 17 | dead space minimization system |
| 18 | first hose |
| 19 | first reservoir unit |
| 20 | first piston pump |
| 21 | first pump unit |
| 22 | first linear motor |
| 23 | first branch |
| 24 | first filter element |
| 25 | second branch |
| 26 | first high pressure sensor |
| 27 | first aeration and bleed valve |
| 28 | first heating element |
| 29 | insulation |
| 30 | control unit |
| 31 | display |
| 32 | first pathway of the dead space minimization system 17 |
| 33 | second pathway of the dead space minimization system 17 |
| 34 | second hose |
| 35 | second reservoir unit |
| 36 | second piston pump |
| 37 | second pump unit |
| 38 | second linear motor |
| 39 | third branch |
| 40 | second filter element |
| 41 | fourth branch |
| 42 | second high pressure sensor |
| 43 | second venting and ventilating valve |
| 44 | second heating element |
| 45 | measuring cuvette |
| 46 | patient set |
| 47 | third hose |
| 48 | ventilation pressure sensor |
| 49a, 49b | opening |
| 50 | connecting pipe |
| 51 | check valve |
| 52 | connecting piece |
| 53 | extension |
| 54 | end of extension 53 |
| 55 | face mask |
| 56 | through-passage |
| 57 | nose of the patient 2 |
| 58 | feed line |
| 59 | nasal prong |
| 60 | outlet of the nasal prong 59 |
| 61 | base body of the through-passage 56 |
| 62 | mask base body |
| 63 | clamping collar |
| 64 | clamping piece |
| 65 | external thread |
| 66 | end of clamping piece 64 |
| 67 | internal thread |
| 68 | diameter taper |
| 69 | longitudinal bead |
| 70 | fixation means |
| 71 | double connector |
| 72 | interface connector |
| 73 | patient hose |

1. A system for supporting pulmonary gas exchange in a patient, comprising:
a ventilating system, which comprises a ventilating device, a Y-piece, a filter, and a patient interface, and comprising
a dead space minimization system that is fluidically coupled to the ventilating system and comprises two separately controllable pump units, each including a reservoir unit and a control unit so that expiratory aspiration of breathing gas and inspiratory recirculation of the aspirated breathing gas by a second of the two pump units can be set, and a first of the two pump units essentially operates in a manner opposite the second pump unit,
the first pump unit being fluidically connected in the region between the ventilating device and the filter, and in particular between the Y-piece and the filter,
wherein the second pump unit is fluidically connected in the region between the filter and the patient interface or, in the case of non-invasive ventilation, can be fluidically connected through the patient interface to the nasal or pharyngeal space of the patient.

2. The system according to claim 1; wherein the ventilating system comprises an aspiration system between the filter and the patient interface, and the second pump unit is fluidically connected in the region between the aspiration system and the patient interface.

3. The system according to claim 1, wherein the second pump unit is fluidically connected to the ventilating system so that a gas flow from the second pump unit joins the ventilating system at an angle of 0° to 70°.

4. The system according to claim 3, wherein a gas flow from the second pump unit joins the ventilating system so that the gas flow from the second pump unit, when joining, has a directional component parallel to a gas flow of the ventilating system toward the ventilating device.

5. The system according to claim 3, wherein a gas flow leaves the ventilating system toward the second pump unit so that the gas flow toward the second pump unit, when leaving, has a directional component parallel to a gas flow of the ventilating system toward the ventilating device.

6. The system according to claim 4, wherein the fluidic connection is carried out via alternating mouths.

7. The system according to claim 1, wherein the second pump unit is fluidically connected to the ventilating system so that a mouth for a gas flow from and to the second pump unit is formed by an appendage protruding into the interior of a connector.

8. The system according to claim 1, characterized in that wherein the patient interface is a face mask or a ventilation helmet for non-invasive ventilation and has a sealing through-passage comprising fixation means for a fluid connection between the nasal or pharyngeal space of the patient and the second pump unit.

9. The system according to claim 1, wherein the patient interface is a face mask or a ventilation helmet for non-invasive ventilation and
comprises an interface connector for connecting a fluid connection to the second pump unit and
a patient hose for the fluidic connection to the nasal or pharyngeal space of the patient.

10. The system according to claim 8, wherein the system comprises a nasal prong and/or an atraumatic catheter for the fluidic connection to the nasal or pharyngeal space of the patient.
